(19) United States
(12) Patent Application Publication
(10) Pub. No.: US 2017/0209464 A1
(43) Pub. Date: Jul. 27, 2017

(54) COMBINATIONS OF FORMOTEROL AND BUDESONIDE FOR THE TREATMENT OF COPD

(71) Applicant: Teva Pharmaceuticals Europe B.V., Amsterdam (NL)

(72) Inventor: Michiel ULLMANN, Amsterdam (NL)

(73) Assignee: Teva Pharmaceuticals Europe B.V., Amsterdam (NL)

(21) Appl. No.: 15/310,130

(22) PCT Filed: May 8, 2015
(86) PCT No.: PCT/EP15/60257

§ 371 Date: Nov. 10, 2016

(30) Foreign Application Priority Data
May 12, 2014 (GB) 1408387.7

(51) Int. Cl. A61K 31/58; A61K 47/26; A61K 9/00; A61K 31/136

(52) U.S. Cl. CPC A61K 31/58; A61K 31/136; A61K 47/26; A61K 9/0075

(57) ABSTRACT

This invention provides a fixed-dose composition comprising formoterol or a pharmaceutically acceptable salt thereof and budesonide, for use in the long-term treatment of COPD and the treatment of acute exacerbations of COPD, wherein the composition is administered as a maintenance dose for the long-term treatment of COPD and pro re nata (p.r.n.) as a rescue medication for the treatment of acute exacerbations of COPD.

The present invention relates to the treatment of respiratory disorders, and particularly to a fixed-dose composition comprising formoterol and budesonide for use in the treatment of chronic obstructive pulmonary disease (COPD).

COPD is a leading cause of death worldwide. Global trends indicate that case frequency will continue to rise and by 2030 COPD will become the fourth leading cause of death worldwide. COPD is considered a preventable and treatable disease and is characterised by persistent airflow limitation that is not fully reversible. The limitation is usually progressive, and primarily associated with an abnormal inflammatory response in the lungs to noxious particles or gases.

COPD is a heterogeneous long-term disease comprising chronic bronchitis, emphysema and also involving the small airways. The pathological changes occurring in patients with COPD are predominantly localised to the airways, lung parenchyma and pulmonary vasculature. Phenotypically, these changes reduce the healthy ability of the lungs to absorb and expel gases.

Bronchitis is characterised by long-term inflammation of the bronchi. Common symptoms may include wheezing, shortness of breath, cough and expectoration of sputum, all of which are highly uncomfortable and detrimental to the patient's quality of life. Emphysema is also related to long-term bronchial inflammation, wherein the inflammatory response results in a breakdown of lung tissue and progressive narrowing of the airways. In time, the lung tissue loses its natural elasticity and becomes enlarged. As such, the efficacy with which gases are exchanged is reduced and respired air is often trapped within the lung. This results in localised hypoxia, and reduces the volume of oxygen being delivered into the patient's bloodstream, per inhalation. Patients therefore experience shortness of breath and instances of breathing difficulty.

Patients living with COPD experience a variety, if not all, of these symptoms on a daily basis. Their severity will be determined by a range of factors but most commonly will be correlated to the progression of the disease. These symptoms, independent of their severity, are indicative of stable COPD and this disease state is maintained and managed through the administration of a variety drugs. The treatments are variable, but often include inhaled bronchodilators, anticholinergic agents, long-acting and short-acting β₂-agonists and corticosteroids. The medicaments are often administered as a single therapy or as combination treatments of corticosteroids and long-acting β₂-agonists.

Stable COPD may be indefinitely maintained, however the disease also manifests itself in an acute form, known in the art as an exacerbation. An exacerbation of COPD is an acute event characterised by a worsening of the patient's respiratory symptoms that is beyond the baseline day-to-day variations and can often lead to a change in medication. Exacerbations may be subcategorised as being mild, moderate or severe, based on, for example, required medications (e.g. oral corticosteroids) and outcomes (e.g. hospitalisation) but are effectively a spectrum of acute worsening of the disorder. Exacerbations can be precipitated by several factors, though it is widely accepted that common causes are respiratory tract infections (viral and bacterial), increased exposure to particulates (air pollution) and poor patient compliance (forgetting or resisting to take medication). These episodes negatively affect the patient's quality of life, accelerate the rate of decline of lung function and are often associated with significant mortality, particularly instances in which hospitalisation is required. During exacerbations patients that seek medical assistance are often treated with short-acting β₂-agonists, corticosteroids and antibiotics, although recent findings have indicated that symptoms persist for several weeks following onset, which suggests that the underlying pathophysiology is not resolved by this approach. Furthermore, it is generally documented that COPD patients frequently experience changeable symptoms. As such, it is estimated that an alarming number of patients endure exacerbations, but choose not to report them, and as a direct result, they suffer irreparable lung damage. These findings highlight an unmet clinical need for improved therapies that manage both stable COPD and offer relief during an exacerbation.

Accordingly, the present invention provides a fixed-dose composition comprising formoterol or a pharmaceutically acceptable salt thereof and budesonide, for use in the long-term treatment of COPD and the treatment of acute exacerbations of COPD, wherein the composition is administered as a maintenance dose for the long-term treatment of COPD and pro re nata (p.r.n.) as a rescue medication for the treatment of acute exacerbations of COPD.

The present invention is based upon a combined treatment of inhaled corticosteroids and β₂-agonists in a single device, which allows patients to receive the benefits of daily maintenance medication and rescue therapy contained within one prescribed dosage (termed a “fixed-dose combination” or “FDC”). Should the patient's symptoms deteriorate (upon experiencing an exacerbation) they will then use the same device as a rescue medication, following secondary (frequency indicating) dosage instructions. Upon multiple actuations of the device, the patient obtains an increased dosage of β₂-agonist that in turn induces bronchodilation and hence provides symptomatic relief. Furthermore, this approach serves to improve patient convenience and compliance through unifying a multi-faceted treatment into a single device. First, the present invention conveniently provides patients with one inhaler to carry, as opposed to two separate inhalers that each contains a different medicament. Secondly, patient compliance is directly addressed and improved, in that, when used as a rescue medication, the patient not only experiences relief from receiving a β₂-agonist but also receives an additional dose of steroid. This feature of the invention is particularly important and beneficial in circumstances where the patient has missed a maintenance dose since it concomitantly provides an increased dose of inhaled corticosteroid to address inflammation that may underlie the worsening of symptoms

It has therefore been found that a combination of budesonide and formoterol may, in a single device, be administered as a maintenance therapy to treat COPD and used also (through increased frequency of actuation) as a rescue medication p.r.n.

Thus, the present invention provides both for the long-term treatment of COPD and the treatment of acute exacerbations of COPD. The long-term treatment involves the administration of a maintenance dose every day. The treatment is typically over a period of more than 6 months, and usually more than 12 months. Many patients will receive the treatment palliatively. This aspect of the disease may be termed “stable COPD”. The acute treatment is for exacerbations, as defined hereinabove. Exacerbations are treated p.r.n., that is, as required. The present invention improves patient care and maintains positive patient prognoses. It particularly provides a therapy that can offer daily symptomatic relief and reduces patient distress in the early stages of, and during, an exacerbation presenting in the home. For this reason, it is often termed a “rescue medication”. It combats persistent inflammation with directed treatment at the appropriate location in the lungs.

Formoterol is a long-acting β₂-agonist that displays a rapid onset of action. It can be synthesised as four independent stereoisomers, and the present invention can include each of these individual forms. Typically it is administered as (R,R)-formoterol, or a racemic mixture of (R,R)- and (S,S)-formoterol. Suitable pharmaceutically acceptable salts of formoterol include those known in the art, and they are commonly derived from the addition of inorganic or organic acids to the medicament. Non-exhaustive examples include hydrochloride, hydrobromide, acetate, formate, halo and alkyl benzoate, tartrate, citrate, fumarate, triflate or salicylate. An example of particular interest is formoterol fumarate, e.g. formoterol fumarate dihydrate.

It is preferable that substantially all of the particles of formoterol fumarate are less than 10 μm in size. This is also to ensure that the particles are effectively entrained in the air stream and deposited in the lower lung, which is the site of action. Preferably, the particle size distribution of the formoterol is d10<1 μm, d50=<5 μm, d90=<10 μm and NLT 99%<10 μm; more preferably, the particle size distribution of the formoterol fumarate is d10<1 μm, d50=1-3 μm, d90=3.5-6 μm and NLT 99%<10 μm.

The delivered dose of formoterol, is preferably 1-20 μg per actuation, with specific examples being 4.5 and 9 μg per actuation. The doses are based on the amount formoterol present (i.e. the amount is calculated without including contribution to the mass of the counterion, where present). The actual prescribed dosage will be dependent upon patient age and weight, severity of disease and response to therapy.

The present invention also comprises the corticosteroid budesonide as a second pharmaceutically active ingredient.

It is preferable that substantially all of the particles of the corticosteroid are less than 10 μm in size. This is to ensure that, when administered with a DPI, the particles are effectively entrained in the air stream and deposited in the lower lung, which is the site of action. Preferably, the particle size distribution of the corticosteroid is d10<1 μm, d50=<5 μm, d90=<10 μm and NLT 99%<10 μm.

The delivered dose of budesonide (the amount actually delivered to the patient) is preferably 50-500 μg per actuation, with specific examples being 80, 160 and 320 μg per actuation. Again, the actual prescribed dosage will be dependent upon patient age and weight, severity of disease and response to therapy.

Particularly preferred delivered doses of budesonide/formoterol in μg are 80/4.5, 160/4.5 and 320/9. Particularly preferred molar ratios of budesonide/formoterol are within the range of 40:1 to 10:1, wherein the moles of formoterol are based on the amount present (i.e. the amount is calculated without including contribution to the mass of the counterion).

The formulation may be administered via inhalation devices known in the art. These can include but are not limited to dry powder inhalers (DPIs) and pressurised metered dose inhalers (pMDIs).

The composition is preferably a dry powder formulation, further comprising a coarse carrier. The carrier can be selected from polysaccharides e.g. glucose or lactose. The carrier is preferably lactose, more preferably lactose monohydrate (α-lactose monohydrate) and may be prepared by standard techniques, e.g. sieving. The lactose carrier preferably has a particle size distribution of d10=20-65 μm, d50=80-120 μm, d90=130-180 μm and <10 μm=<10%. Preferably, the particle size distribution of the lactose is d10=20-65 μm, d50=80-120 μm, d90=130-180 μm and <10 μm=<6%.

A suitable inhaler for working the present invention is the Spiromax® DPI available from Teva Pharmaceuticals.

The delivered dose of the active agent is measured as per the USP <601>, using the following method. A vacuum pump (MSP HCP-5) is connected to a regulator (Copley TPK 2000), which is used for adjusting the required drop pressure P₁ in a DUSA sampling tube (Dosage Unit Sampling Apparatus, Copley). The inhaler is inserted into a mouthpiece adaptor, ensuring an airtight seal. P₁ is adjusted to a pressure drop of 4.0 KPa (3.95-4.04 KPa) for the purposes of sample testing. After actuation of the inhaler, the DUSA is removed and the filter paper pushed inside with the help of a transfer pipette. Using a known amount of solvent (acetonitrile:methanol:water (40:40:20)), the mouthpiece adaptor is rinsed into the DUSA. The DUSA is shaken to dissolve fully the sample. A portion of the sample solution is transferred into a 5 mL syringe fitted with Acrodisc PSF 0.45 μm filter. The first few drops from the filter are discarded and the filtered solution is transferred into a UPLC vial. A standard UPLC technique is then used to determine the amount of active agent delivered into the DUSA. The delivered doses of the inhaler are collected at the beginning, middle and end of inhaler life, typically on three different days.

In one embodiment the composition is administered 2-4 times per day as a maintenance dose, more preferably the composition is administered twice-per-day (i.e. b.i.d.) as a maintenance dose. B.i.d. administration is typically every morning and every evening as a maintenance dose and the required dose may be administered in one or two puffs of the inhaler.

The composition is preferably administered no more than ten times p.r.n as a rescue medication, more preferably no more than eight times p.r.n as a rescue medication. In a particularly preferred embodiment, the composition is administered twice-per-day as a maintenance dose and no more than eight times p.r.n as a rescue medication. Ideally, the patient should not exceed 120 μg of formoterol over any 24 hour period and 3,200 μg of budesonide over any 24 hour period.

The present invention will now be described with reference to the examples, which are not intended to be limiting.

### EXAMPLES

### Example 1

Three formulations of Budesonide/Formoterol (BF) Spiromax (Teva Pharmaceuticals) were prepared: low strength (120 inhalations, each delivering 80 μg budesonide and 4.5 μg formoterol), middle strength (120 inhalations, 160 μg budesonide and 4.5 μg formoterol per inhalation), and high strength (60 inhalations, 320 μg budesonide and 9 μg formoterol per inhalation).

The compositions of the three strengths of BF Spiromax per container are set out in Tables 1-3.

TABLE 1
Composition per container of BF Spiromax 80/4.5 μg 120 inhalation product

| Material | Weight | | Function | Quality Standard |
|---|---|---|---|---|
| Budesonide (micronised) | 12.0 | mg | Drug | Ph. Eur. |
| | | | substance | |
| Formoterol fumarate dihydrate | 0.645 | mg | Drug | Ph. Eur. |
| (micronised) | | | substance | |
| Lactose monohydrate | 1.487 | g | Excipient | Ph. Eur. |
| Target fill weight per device | 1.500 | g | | |

TABLE 2
Composition per Container of BF Spiromax 160/4.5 μg 120 inhalation product

| Material | Weight | | Function | Quality Standard |
|---|---|---|---|---|
| Budesonide (micronised) | 31.6 | mg | Drug | Ph. Eur. |
| | | | substance | |
| Formoterol fumarate dihydrate | 0.914 | mg | Drug | Ph. Eur. |
| (micronised) | | | substance | |
| Lactose monohydrate | 0.838 | g | Excipient | Ph. Eur. |
| Target fill weight per device | 0.870 | g | | |

TABLE 3
Composition per Container of BF Spiromax 320/9 μg 60 inhalation product

| Material | Weight | | Function | Quality Standard |
|---|---|---|---|---|
| Budesonide (micronised) | 28.7 | mg | Drug | Ph. Eur. |
| | | | substance | |
| Formoterol fumarate dihydrate | 0.870 | mg | Drug | Ph. Eur. |
| (micronised) | | | substance | |
| Lactose monohydrate | 0.840 | g | Excipient | Ph. Eur. |
| Target fill weight per device | 0.870 | g | | |

### Example 2

This is a two-arm parallel study investigating whether symptom-driven maintenance and reliever/rescue therapy with budesonide/formoterol is more effective as a single device dual treatment regimen that manages and also concomitantly reduces the number of exacerbations of COPD compared to a multiple device fixed maintenance dose of fluticasone/salmeterol and salbutamol as a rescue medication.

Patient Group A (Invention)

Participants are receiving Spiromax® budesonide/formoterol 160/4.5 μg, two inhalations, twice daily and additionally, Spiromax® budesonide/formoterol 160/4.5 μg as needed, with a maximum of eight additional inhalations per day for rescue use.

Patient Group B (Comparative)

Participants are receiving Diskus® fluticasone/salmeterol (steroid/long-acting β₂-agonist) 500/50 μg, one inhalation, twice daily and additionally, salbutamol (short-acting β₂-agonist) 100 μg as needed with a maximum of eight additional inhalations per day. The comparative study represents an example of the current standard treatment for COPD.

Patients are being subjected to constant evaluation throughout the investigation. Key parameters that are being assessed include; but are not limited to, reduction in the number of exacerbations (moderately severe and severe exacerbations combined), reductions in hospitalisation during exacerbations, improvement in patient compliance and convenience, general lung function (PEF, FEV1, FEV1/FVC, FEV25-75%, RV, TLC, RV/TLC, RV/TLC %, predicted).

1. A method of long-term treatment of COPD and the treatment of acute exacerbations of COPD in a patient, comprising administering to the patient a fixed-dose composition comprising formoterol or a pharmaceutically acceptable salt thereof and budesonide, wherein the composition is administered as a maintenance dose for the long-term treatment of COPD and pro re nata (p.r.n.) as a rescue medication for the treatment of acute exacerbations of COPD.

2. The method of claim 1, wherein the composition is a dry powder formulation further comprising a coarse carrier.

3. The method of claim 2, wherein the coarse carrier is lactose.

4. The method of claim 1, wherein formoterol is present in said fixed-dose composition as formoterol fumarate.

5. The method of claim 1, wherein the delivered dose of formoterol, based on the amount of formoterol, is 1-20 μg.

6. The method of claim 1, wherein the delivered dose of budesonide is 5-500 μg.

7. The method of claim 1, wherein the delivered doses of formoterol/budesonide in μg are 80/4.5, 160/4.5 or 320/9.

8. The method of claim 1, wherein the composition is administered 2-4 times per day as a maintenance dose.

9. The method of claim 7, wherein the composition is administered twice-per-day as a maintenance dose.

10. The method of claim 1, wherein the composition is administered no more than ten times p.r.n as a rescue medication.

11. The method of claim 9, wherein the composition is administered no more than eight times p.r.n as a rescue medication.

12. The method of claim 1, wherein the composition is administered twice-per-day as a maintenance dose and no more than eight times p.r.n as a rescue medication.
